(19) United States
(12) Patent Application Publication
(10) Pub. No.: US 2005/0187462 A1
(43) Pub. Date: Aug. 25, 2005

(54) Dynamic contrast enhanced imaging using a mamillary distributed parameter model

(76) Inventors: Tong San Koh, Singapore (SG); Lai Hong Dennis Cheong, Singapore (SG)

Correspondence Address: DINSMORE & SHOHL LLP, Sutie 500, One Dayton Centre, Dayton, OH, 45402-2023, US

(21) Appl. No.: 10/768,451

(22) Filed: Jan. 30, 2004

(51) Int. Cl. A61B 5/055

(52) U.S. Cl. 600/416

(57) ABSTRACT

A method of dynamic contrast enhanced (DCE) imaging includes: for a region of interest, sampling the concentration of a tracer liquid in a fluid over a time interval to arrive at a plurality of samples; fitting the plurality of samples to a plurality of multi-compartment mamillary pharmacokinetic models, each of the plurality of models modelling fluid flow in a number of interstitial compartments about a central compartment at the region interest, as a result of flow caused by a source of the fluid, so as to determine a plurality of fitting parameters for each of the plurality of models; determining a preferred one of the plurality of models; and presenting indicators indicative of values of the plurality of parameters for the preferred one of the models. Numerous multi-compartment mamillary pharmacokinetic models are disclosed. The models and methods may be used in DCE imaging systems and software.

### FIELD OF THE INVENTION

The present invention relates generally to medical imaging, and more particularly to dynamic contrast enhanced imaging, using a mamillary distributed parameter model.

### BACKGROUND OF THE INVENTION

In recent years, numerous new medical imaging techniques have been introduced, and have advanced significantly. Techniques such as magnetic resonance imaging (MRI), computed tomography (CT), ultrasound, radio isotope imaging, and electrical impedance tomography have joined conventional x-ray imaging.

Dynamic contrast-enhanced magnetic (DCE) imaging is based on the acquisition of images, using for example MRI or CT scanning, in the presence of a contrast medium.

Typically, images in a region of interest over a time interval before, during, and after the administration of a contrast medium are acquired. Acquired data is then fitted to pharmacokinetic models from which parameters based on the rates of fluid exchange can be calculated, as for example detailed in P. S. Tofts, G. Brix, D. L. Buckley, J. L. Evelhoch, E. Henderson, M. V. Knopp, H. B. W. Larsson, T. Lee, N. A. Mayr, G. J. M. Parker, R. E. Port, J. Taylor, and R. M. Weisskoff, “Estimating kinetic parameters from dynamic contrast-enhanced T1-weighted MRI of a diffusible tracer: standardized quantities and symbols”, JMRI, vol. 10, pp. 223-232, 1999.

By fitting the acquired data to a pharmacokinetic model, parametric values may be displayed to allow a fuller depiction of the wash-in and wash-out contrast kinetics of the contrast medium within tumours, than conventional (i.e. non-dynamic) imaging techniques. DCE imaging thus provides insight into the nature of the bulk tissue properties.

Current pharmacokinetic models model fluid flow in tissue in compartments. Multiple compartment pharmacokinetic models can be classified according to (a) the nature of the compartments, i.e as conventional compartmental and distributed-parameter; and (b) the structure and interactions of the various compartments, i.e as catenary or mamillary. In catenary models, compartments are modelled as connected in a linear array with each compartment communicating with its closest neighbour. In mamillary models, a central compartment is modelled as communicating with one or more peripheral compartments. For conventional compartmental models, both the catenary and mamillary multi-compartment models have been previously proposed. For the distributed parameter models, 2- and 3-compartment catenary models have, for example, been proposed in K. B. Larson, J. Markham, and M. E. Raichle, “Tracer-kinetic models for measuring cerebral blood flow using externally detected radiotracers”, J. Cerebral Blood Flow and Metabolism, vol. 7, pp. 443-463, 1987.

Calculated parameters can be used to generate color-encoded images that aid in the visual assessment of tumors, as for example detailed in PCT patent publication WO 03/00710. DCE imaging is currently used to characterize masses, stage tumors, and non-invasively monitor therapy.

While current DCE imaging techniques are in clinical use, they have numerous limitations, including overlap between malignant and benign inflammatory tissue, failure to resolve microscopic disease, and the inconsistent predictive value of enhancement pattern with regard to clinical outcome. In part, some of these limitations are attributable to the pharmacokinetic models used in current DCE imaging techniques.

Accordingly, there is a need for new DCE imaging techniques and apparatus, employing enhanced pharmacokinetic models.

### SUMMARY OF THE INVENTION

In accordance with an aspect of the present invention, a method of DCE imaging includes: for a region of interest, sampling the concentration of a tracer liquid in a fluid over a time interval to arrive at a plurality of samples; fitting the plurality of samples to a plurality of multi-compartment mamillary pharmacokinetic models, each of the plurality of models modelling fluid flow in a number of interstitial compartments about a central compartment at the region interest, as a result of flow caused by a source of the fluid, so as to determine a plurality of fitting parameters for each of the plurality of models; determining a preferred one of the plurality of models; presenting indicators indicative of values of the plurality of parameters for the preferred one of the models.

In accordance with another aspect of the present invention, a plurality of samples of concentration of a tracer liquid in a fluid over time are fitted to a multi-compartment mamillary pharmacokinetic model modelling fluid flow in a number of interstitial compartments about a central compartment at the region interest, as a result of flow caused by a source of the fluid. The pharmacokinetic model has an impulse residue response function that may be expressed in the form,

$$\begin{matrix}{R}_{n} ( t ) = {L}_{t}^{- 1} [ \frac{1}{s} ] - exp ( - {χ}_{n} ) {L}_{t}^{- 1} [ exp ( - {t}_{1} s ) \frac{1}{s} {\prod }_{i = 2}^{n} exp ( \frac{{k}_{i}}{s + {k}_{1 i}} ) ] \\ = u ( t ) - exp ( - {χ}_{n} ) u ( t - {t}_{1} ) \\ {\int }_{0}^{t - {t}_{1}} ( {y}_{2} ⊗ ⋯ ⊗ {y}_{i} ⊗ ⋯ ⊗ {y}_{n} ) ⅆ τ wherein \\ {y}_{i} ( t ) = {L}_{t}^{- 1} [ exp ( \frac{{k}_{i}}{s + {k}_{1 i}} ) ] \\ = exp ( - {k}_{1 i} t ) {δ}_{t} ( t ) + exp ( - {k}_{1 i} t ) \sqrt{\frac{{k}_{i}}{t}} {I}_{1} ( 2 \sqrt{{k}_{i} t} ) ;\end{matrix}$$

I₁ is the modified Bessel function; u(t) is the Heaviside unit step function, δₜ is the dirac delta function; n is the number of compartments for the model, k₁ⱼ is a transfer constant denoting the rate of permeation from the central compartment to the nth interstitial compartment, per unit volume of the capillary,

$${χ}_{n} = {t}_{1} {\sum }_{i = 2}^{n} {k}_{i1} , and {k}_{i} = {t}_{1} {k}_{1 i} {k}_{i1} .$$

These models and methods may be used in otherwise conventional DCE imaging systems, or embodied in software.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures which illustrate by way of example only, embodiments of the present invention,

FIG. 1 is a schematic diagram of a multi compartment, mamillary capillary model used in embodiments of the present invention;

FIG. 2 is a flow chart, illustrating steps performed by a dynamic contrast enhancement imaging device, in manners exemplary of the present invention;

FIG. 3A-3C are images formed by DCE imaging device in manners exemplary of embodiments of the present invention;

FIG. 4 illustrates measured blood flow from an arterial source and tracer concentration in a region of interest, in an example application of the method of FIG. 2;

FIG. 5 illustrates measured tracer concentration data in a region of interest, and the fitting of a plurality models to the data, in an example application of the method of FIG. 2; and

FIG. 6 illustrates the impulse response function corresponding to the plurality of models of FIG. 5.

### DETAILED DESCRIPTION

As noted, DCE imaging typically involves the intravenous introduction of a contrast medium (or tracer) in the fluid blood stream and subsequent sequential imaging to simultaneously monitor the course of the tracer concentration in the tissue in a region of interest and the feeding artery (or fluid source) over time.

For example, for CT imaging, tracer concentration in a region of interest can be estimated by averaging the sensed concentration in a region of interest. For example, the tracer used in CT imaging is commonly iodine based. In the CT image, the intensity value is directly proportional to the concentration of tracer. For MRI, other techniques may be employed: known transforms correlate image intensity and tracer concentration.

Mathematically, a relationship between tracer flow in the tissue Cₜᵢₛₛ(t) at a region of interest, and the feeding artery Cₐ(t) can be derived by assuming a causal, linear time-invariant (LTI) system. The response of the system (residual tracer in the system) due to an arbitrary input can then be derived with the use of an impulse residue response function and the convolution operator \mathring{x}. The operational equation expressing the relationship between Cₜᵢₛₛ(t) and Cₐ(t) then takes the form of a convolution integral:

$${C}_{tiss} ( t ) = \frac{F ρ}{1 - Hct} {C}_{a} ( t0 ⊗ {R}_{n} ( t ) \qquad ( 1 )$$

where the fractional hematocrit, Hct, is the volume fraction of whole blood taken up by cells, F is the perfusion or blood flow and ρ is the density of the tissue. Here, the fractional hematocrit Hct is the volume fraction of whole blood taken up by cells and is typically approximated. For major vessels, Hct is typically approximated as about 0.45, as detailed in P.S. Tofts et al, supra.

Rₙ(t) is thus the impulse residue response function. An appropriate model of Rₙ(t) allows quantification of the underlying microcirculatory parameters. This is for example, detailed in P. Meier and K. L. Zieler, “On the theory of the indicator-dilution method for measurement of blood flow and volume”, J. Appl. Physiol., vol. 6, pp. 731-744, 1954, and K. L. Zierler, “Equations for measuring blood flow by external monitoring of radioisotopes”, Cir. Res., vol. XVI, pp. 309-321, 1965.

Exemplary of an embodiment of the present invention, n-compartment mamillary distributed-parameter models for capillary-tissue exchange are proposed for DCE imaging. The multiple compartments model the tissue's heterogeneity.

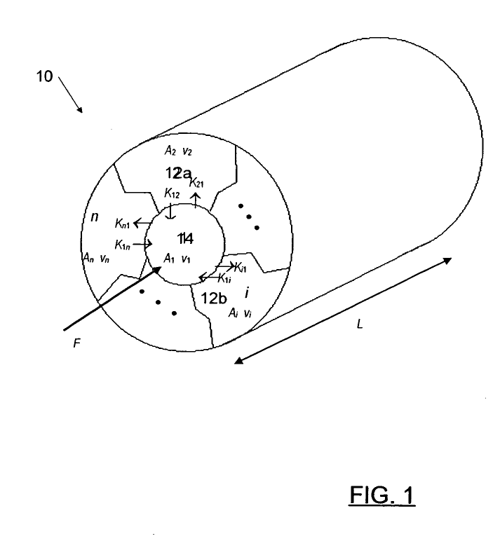

A schematic diagram of such a capillary-tissue model is shown in FIG. 1. This hypothetical unit capillary-tissue model can be assumed to represent an average of the underlying vasculature. In the depicted mamillary system 10, tissue is modelled by a number (n-1) of peripheral interstitial compartments 12a, 12b, 12c . . . (collectively and individually compartments 12) around the central vascular compartment 14.

The single vascular compartment 14 partakes in diffusive exchange with n-1 surrounding interstitial compartments 12 while exchange between interstitial compartments is assumed to be zero. The rate of transfer from the j-th compartment to the i-th compartment is denoted by Kᵢⱼ. The central vascular compartment 14 is labeled with index i=1, while i=2, . . . ,n denote the peripheral interstitial compartments 12. Direction of blood flow F is along the length of the capillary, L. Each compartment 12, 14 is further characterized by a cross-sectional area Aᵢ and fractional volume vᵢ. Concentration in the vascular compartment is a function of both time and position while interstitial concentrations are functions of only time.

Considering mass conservation of tracer for elemental volume, the following set of partial differential equations can be derived for the mamillary system 10:

$$\begin{matrix}{v}_{1} \frac{\partial {C}_{1}}{\partial t} + F ρ L \frac{\partial {C}_{1}}{\partial z} + {\sum }_{i = 2}^{n} {K}_{i1} {C}_{i} - {\sum }_{i = 2}^{n} {K}_{1 i} {C}_{i} = L {δ}_{t} ( t + 0 ) {δ}_{z} ( z + 0 ) & ( 2 a ) \\ {v}_{i} \frac{ⅆ {C}_{i}}{ⅆ t} + {K}_{1 i} {C}_{i} - {K}_{il} {C}_{i} = 0 ( i = 2 , … n ) . & ( 2 b )\end{matrix}$$

L is the length of the capillary; F is the flow rate of blood/fluid; ρ is the density of the fluid/blood; and the Dirac delta functions, δₜ and δ_{z} denote the idealized impulse excitation of a unit-mass source with dimensions of reciprocal time and reciprocal length respectively. Cᵢ denotes the concentration in the i-th compartment. Kᵢⱼ is the transfer constant and denotes the rate of permeation from the j-th compartment to the i-th compartment. vᵢ is the fractional volume (i.e volume of the i-th compartment per unit volume of tissue) which is dimensionless.

The generalized form of the impulse residue response function for a n-compartment mamillary distributed parameter model can thus be written as

$$\begin{matrix}{R}_{n} ( t ) = {L}_{t}^{- 1} [ \frac{1}{s} ] - exp ( - {χ}_{n} ) {L}_{t}^{- 1} [ exp ( - {t}_{1} s ) \frac{1}{s} {\prod }_{i = 2}^{n} exp ( \frac{{k}_{i}}{s + {k}_{1 i}} ) ] \\ = u ( t ) - exp ( - {χ}_{n} ) u ( t - {t}_{1} ) \\ {\int }_{0}^{t - {t}_{1}} ( {y}_{2} ⊗ ⋯ ⊗ {y}_{i} ⊗ ⋯ ⊗ {y}_{n} ) ⅆ τ . \end{matrix} \qquad ( 3 )$$

Here L⁻¹ is the inverse Laplace transform and u(t) is the Heaviside unit step function, and

$$\begin{matrix}{y}_{i} ( t ) = {L}_{t}^{- 1} [ exp ( \frac{{k}_{i}}{s + {k}_{1 i}} ) ] \\ = exp ( - {k}_{1 i} t ) {δ}_{t} ( t ) + exp ( - {k}_{1 i} t ) \sqrt{\frac{{k}_{i}}{t}} {I}_{1} ( 2 \sqrt{{k}_{i} t} )\end{matrix} \qquad ( 4 )$$

where I₁ is the modified Bessel function, and

$$\begin{matrix}{χ}_{n} = {t}_{1} {\sum }_{i = 2}^{n} {k}_{i1} , and & ( 5 ) \\ {k}_{i} = {t}_{1} {k}_{1 i} {k}_{i1} . & ( 6 )\end{matrix}$$

χₙ is associated with the extravasation or leaking of tracer from the vascular region to the interstitial regions. Hence it is a kinetic parameter of the model. With

$${k}_{ij} = \frac{{K}_{ij}}{{v}_{j}} \qquad ( 7 )$$

where vⱼ is the fractional volume of the j-th compartment, Kᵢⱼ is the transfer constant from j-th compartment to the i-th compartment, and kᵢⱼ is defined as the rate constant for tracer transfer from compartment j to compartment i.

So, for a 2-compartment mamillary distributed parameter model,

$$\begin{matrix}{R}_{2} ( t ) = u ( t ) - exp ( - {χ}_{2} ) u ( t - {t}_{1} ) [ 1 + {X}_{2} ( t - {t}_{1} ) ] \\ where {X}_{i} ( t ) = {\int }_{0}^{t} {x}_{i} ( τ ) ⅆ τ and & ( 8 a ) \\ {x}_{i} ( t ) = exp ( - {k}_{1 i} t ) \sqrt{\frac{{k}_{i}}{t}} {I}_{1} ( 2 \sqrt{{k}_{i} t} ) . & ( 8 b )\end{matrix}$$

For a 3-compartment model,

$$\begin{matrix}{R}_{3} ( t ) = u ( t ) - exp ( - {χ}_{3} ) u ( t - {t}_{1} ) [ 1 + {\sum }_{i = 2}^{3} {X}_{i} ( t - {t}_{1} ) + {W}_{23} ( t - {t}_{1} ) ] \\ where & ( 9 a ) \\ {W}_{ij} ( t ) = {\int }_{0}^{t} ( {x}_{i} ⊗ {x}_{j} ) ⅆ τ . & ( 9 b )\end{matrix}$$

An expression for the 4-compartment mamillary distributed parameter model may be written as

$$\begin{matrix}\begin{matrix}{R}_{3} ( t ) = u ( t ) - exp ( - {χ}_{4} ) u ( t - {t}_{1} ) \\ \times [ 1 + {\sum }_{i = 2}^{4} {X}_{i} ( t - {t}_{1} ) + {W}_{23} ( t - {t}_{1} ) + {W}_{34} ( t - {t}_{1} ) \\ \times + {W}_{42} ( t - {t}_{1} ) + {U}_{234} ( t - {t}_{1} ) ]\end{matrix} \\ where & ( 10 a ) \\ {U}_{ijk} ( t ) = {\int }_{0}^{′} ( {x}_{i} ⊗ {x}_{j} ⊗ {x}_{k} ) ⅆ τ . & ( 10 b )\end{matrix}$$

Expressions for five (5) or more compartment mamillary models may be similarly derived in manners understood by those of ordinary skill.

So, for any set of acquired imaging data, the generalized form of the impulse residue response function for the multiple compartment mamillary distributed parameter model, allows an approximation of a number of kinetically distinct compartments for any particular set of data using conventional statistical testing.

To test whether the observed data admits additional compartments, thereby demonstrating tissue kinetic heterogeneity, the data set may be fit using the proposed models with increasing number of compartments. A preferred model would be one that is the simplest (i.e. includes as few compartments as possible) and whose goodness-of-fit is not significantly worse than the more complex (i.e. having more compartments) ones.

The goodness of fit for each model may be assessed by

$${χ}^{2} = {\sum }_{k = 1}^{n} {[ \frac{{C}_{tiss}^{exp 1} - {C}_{tiss} ( F , {t}_{1} , {k}_{ij} , … )}{{var}_{k}} ]}^{2} \qquad ( 11 )$$

where varₖ represents the variance of the sampled data points.

A test statistic to compare the goodness-of-fit of a more complex model (χ²_{c}) with that of a less complex (reduced) model (χ²ᵣ), can be given by

$${F}^{*} ( {χ}_{r}^{2} , {χ}_{c}^{2} ) = \frac{\frac{{χ}_{r}^{2} - {χ}_{c}^{2}}{{df}_{r} - {df}_{c}}}{\frac{{χ}_{c}^{2}}{{df}_{c}}} \qquad ( 12 )$$

which follows the F-distribution. The degrees of freedom dfᵣ and df_{c} are those associated with the reduced and complex models, respectively. The degrees of freedom may be calculated as df=(number of datapoints−number of parametres−1). Since χ²ᵣ≧χ²_{c}, a one-tailed F-test at significance level α=0.05 may be appropriate.

When the number of kinetically distinct compartments is determined, the preferred model can be used to simultaneously derive several useful haemo-dynamic parameters associated with the underlying tissue, such as perfusion, blood volume and transit time, permeability, extraction ratio and transfer constants of the various compartments.

For each fitting, two types of parameters may be determined: those that are directly obtained through fitting are called fundamental parameters (i.e F, t₁, k₂ᵢ, k₁₂, . . . kₙ₁, k₁ₙ, total=2n), and other parameters that can be calculated from the fundamental parameters (e.g. E, v₁, v₂ . . . vₙ, and Kᵢⱼ=PSᵢⱼ). Both types of parameters are useful in the study and characterization of tumors.

For example, the transfer constant between compartments Kᵢⱼ can be associated with the permeability surface area product (PS) between blood plasma, assuming Kᵢ₁=PSᵢ₁, where PSᵢ₁ is the permeability surface area product between the vascular compartment (compartment 1) and the i-th compartment.

Similarly, the parameter F indicative of blood flow along the length of the capillary is indicative of blood perfusion.

Such parameters possess diagnostic value as they can be used to reflect haemo-dynamic abnormalities of the tissue and when quantified, can serve as potential indicators of pathologies.

The model can also be used to describe the haemo-dynamics and capillary-tissue exchange for tissues that exhibit kinetic heterogeneity, which include various types of tumors in organs such as the brain, breast or prostate etc.

The physical significance of model parameters may be appreciated by noting that the impulse residue response functions associated with distributed-parameter models can be written in a separable form in time domain, with each component representing a physiologic process.

For the present mamillary model,

$$\begin{matrix}{R}_{n} ( t ) = {R}_{n}^{v} ( t ) + {R}_{n}^{p} ( t ) \\ where & ( 13 ) \\ {R}_{n}^{v} ( t ) = \{ \begin{matrix}1 & 0 < t \leq {t}_{1} \\ 0 & t > {t}_{1}\end{matrix} & ( 14 a )\end{matrix}$$

represents the vascular phase and

$${R}_{n}^{p} ( t ) = \{ \begin{matrix}0 & 0 < t \leq {t}_{1} \\ 1 - exp ( - {χ}_{n} ) {\int }_{0}^{- {t}_{1}} ( {y}_{2} ⊗ … ⊗ {y}_{n} ) ⅆ τ & t > {t}_{1}\end{matrix} \qquad ( 14 b )$$

denotes the clearance of tracer from the parenchyma tissue i.e the parenchyma clearance phase. This is a convenient property of the distributed-parameter models since it can readily be used to illustrate the importance of each component phase. For a strictly intravascular tracer, the parenchyma component vanishes and the impulse residue function is fully characterized by the vascular component; while for a largely extravascular tracer, a significant portion of the impulse residue function would be non-zero after t₁. The ability of a model to reflect such situations is beneficial as the vessel permeability is a property of both the tracer and tissue concerned. Conventional clinical contrast medium of MRI and CT (with molecular weight of ˜600 daltons) rapidly diffuse through capillary walls of both normal and tumoral vessels. However, recent trends in developing macro-molecular contrast medium (MMCM) has resulted in blood pool contrast agents (such as Albumin-Gd-DTPA35 of ˜92,000 daltons) which diffuse very slowly, if at all, through normal endothelial walls. Such MMCM developed for both MRI and CT have molecular sizes that approximate those of serum proteins and can be used to study hypervascularity and hyperpermeability of tumors.

Several important physiological parameters can be formerly evaluated. For example, as the value of Rₙ(t) in the limit t approaches t₁⁺ in equation (14b), the fractional extraction constant E can again be written as

$$E = {lim}_{t -> {t}_{1} +} {R}_{n}^{p} ( t ) = 1 - exp ( - {χ}_{n} ) = 1 - exp ( - {t}_{1} {\sum }_{i = 2}^{n} {k}_{i1} ) , \qquad ( 15 )$$

which is a function of the fitted parameters t₁ and kᵢ₁ . . . kₙ₁. As will be appreciated, the fractional extraction constant E is indicative of the extraction of tracer from the vascular space (i.e. central compartment) to the interstitial space (i.e. interstitial compartments) and may be used to quantify the leakiness of the capillaries.

Similarly, the fractional vascular volume can be calculated from the flow rate and vascular transit time using v₁=Ft₁ (taking ρ=1). This relation can also be inferred using the central volume principle, as, for example, detailed in P. Meier and K. L. Zieler, “On the theory of the indicator-dilution method for measurement of blood flow and volume”, J. Appl. Physiol., vol, 6, pp. 731-744, 1954, and K. L. Zierler, “Equations for measuring blood flow by external monitoring of radioisotopes”, Cir. Res., vol. XVI, pp. 309-321, 1965. The transfer constant Kᵢ₁ can then be estimated from the corresponding rate constant kᵢ₁ using equation (16).

Likewise, it may be noted that K₁ᵢ and Kᵢ₁ in general differ by the partition coefficient of compartments involved, as detailed in P. S. Tofts, G. Brix, D. L. Buckley, J. L. Evelhoch, E. Henderson, M. V. Knopp, H. B. W. Larsson, T. Lee, N. A. Mayr, G. J. M. Parker, R. E. Port, J. Taylor, and R. M. Weisskoff, “Estimating kinetic parameters from dynamic contrast-enhanced T1-weighted MRI of a diffusible tracer: standardized quantities and symbols”, JMRI, vol. 10, pp. 223-232, 1999; K. S. St. Lawrence, and T. Lee, “An adiabatic approximation to the tissue homogeneity model for water exchange in the brain: I. Theoretical derivation”, J. Cereb. Blood Flow and Metabolism, vol. 18, pp. 1365-1377, 1998; and K. L. Zierler, “Equations for measuring blood flow by external monitoring of radioisotopes”, Cir. Res., vol. XVI, pp. 309-321, 1965. For an easily diffusible tracer, the assumption of unit partition coefficient is made in most perfusion imaging studies, such that the transfer constant in opposite directions are often assumed to be equal, i.e. Kᵢ₁=K₁ᵢ=Kᵢ. Using this assumption of equal transfer constants in both directions, the corresponding fractional volume associated with each of the i-th peripheral interstitial compartment may be estimated by

$${v}_{i} = {v}_{1} \frac{{k}_{i 1}}{{k}_{1 i}} . \\ with {v}_{1} = {Ft}_{1} . \qquad ( 16 )$$

The ability of the present models to allow for simultaneous estimation of these microcirculatory parameters is of importance considering recent studies which show that these parameters can be potentially useful in differentiating tumors. In studies of breast lesions detailed in C. A. Hulka, B. L. Smith, D. C. Sgroi, L. Tan, W. B. Edmister, J. P. Semple, T. Campbell, D. B. Kopans, T. J. Brady, and R. M. Weisskoff, “Benign and malignant breast lesions: differentiation with echo-planar MR Imaging”, Radiology, vol. 197, pp. 33-38, 1995, and C. A. Hulka, W. B. Edmister, B. L. Smith, L. Tan, D. C. Sgroi, T. Campbell, D. B. Kopans, and R. M. Weisskoff, “Dynamic echo-planar imaging of the breast: experience in diagnosing breast carcinoma and correlation with tumor angiogenesis”, Radiology, vol. 205, pp. 837-842, 1997, for example, it was found that the extraction-flow product (an estimate of the transfer constant v in a mixed F- and PS-limited model is higher for malignant than benign lesions, while normal parenchyma clearly has much lower extraction-flow products. Also, as gliomas have regions with higher vascularization than in healthy brain tissues, fractional vascular and interstitial volume maps have been proposed to be used as a tool for differentiating between brain tumors as it was found that vascular volume correlated closely with histologic tumor grading.

The model defined in equation (3), supra, assumes that the tracer kinetics in the region of interest can be modelled by an averaged capillary-tissue unit. An alternative model may take into account the distribution of capillary transit times in individual adjacent capillaries.

With this formulation, one can further quantify an additional parameter associated with the variance of the capillary transit time spectrum/distribution, which can be used to assess the degree of randomness, or flow heterogeneity within the tumor (as different from kinetic heterogeneity which is associated with the number of kinetically different compartments).

Let g(t₁) denote the distribution of vascular transit times for the multiple capillary pathways through the tissue. For g(t₁) to be physiologically realistic and acceptable, it should be non-negative, casual, skewed (i.e asymmetric) and normalized.
∫₀^{∞}g(τ)dτ=1 (17)

Using the distributed parameter model of equation (3) to describe the transit of tracer through each capillary pathway, the total contribution due to all the capillaries in the vasculature can be summed in the usual statistical sense. The overall impulse residue function of the vasculature can again be given in terms of contributions due to the two physiologic phases,
Hₙ(t)=Hₙ^{v}(t)+Hₙ^{p}(t) (18a)
where
Hₙ^{v}(t)=1−∫₀dt₁g(t₁) (18b)
and
Hₙ^{p}(t)=∫₀dt₁g(t₁)Rₙ^{p}(t,t₁) (1 8c)

denotes the vascular and parenchyma phases,

where yₙ is defined in equation (4), and g(t₁) denotes the distribution of the capillary transit times. Any functional from which satisfy non-negative, casual, skewed (i.e asymmetric) and normalized can used as g(t₁).

In this form, the resulting function is continuous and hence physiologically more realistic than equation (3). Functionally admissible forms of g(t) should account for the left-skewed nature of blood vessel distribution in tumors. Although a distribution function includes two parameters (mean μ and standard deviation σ) the final form of the model given by equation (18a) has only one parameter more than the corresponding 2n parameters of the mamillary distributed parameter model.

A plausible distribution function for the capillary transit time spectrum is the delayed gamma variate with constrained skewness,

$${g}_{Γ} ( τ ) = \frac{1}{N} {τ}^{2} exp ( - b τ ) ⊗ δ ( τ - {t}_{d} ) \qquad ( 19 )$$

where the adjustable parameters are, b which controls the shape of the gamma variate, and delay time t_{d} which specifies the minimum vascular transit time, when gᵣ(τ) is used to denote the transit time spectrum. gᵣ(τ) is normalized with N=2/b³, and the mean and standard deviation (dispersion) of the vascular transit times are respectively, μ=t_{d}+3/b, and σ=\sqrt{3}/b.

The additional parameter is the standard deviation of the distribution function g(t₁). That is, for any distribution function g(t₁), although one can define a mean μ and standard deviation, σ, the μ replaces the original t₁ (which is the average transit time of the hypothetical capillary unit) in the model of equation (3), requiring only the additional parameter σ.

As well, compared to the recently proposed distributed capillary adiabatic tissue homogeneity model detailed in T. S. Koh, L. H. Cheong, Z. Hou, and Y. C. Soh, “A physiologic model of capillary-tissue exchange for dynamic contrast-enhanced imaging of tumor microcirculation”, IEEE Trans. Biomed. Eng., vol. 50:159-167, February 2003, the contents of which are hereby incorporated by reference, this model has the same number of parameters, but may possess more realism as it do not assume the approximations needed for the adiabatic model.

The rationale for including a statistical parameter for variance into the impulse response model is also the fact that tumor capillaries are randomly formed (through angio-neogenesis), and such a parameter can allow for the quantification of the degree of randomness. Possibly, the variance parameter, together with the kinetic parameters in the mamillary model, can be used for quantification and classification of the patho-physiological states of the tumor.

The above described models may easily be implemented in otherwise conventional imaging equipment (such as MRI or CT systems). Software exemplary of embodiments of the present invention may be loaded in such systems to allow imaging in manners exemplary of the present invention. For example, exemplary methods may be embodied in a GE Medical Systems HiSpeed CT imaging system. A person of ordinary skill will of course readily appreciate that most conventional medical imagers capable of continuous scanning mode may be adequate.

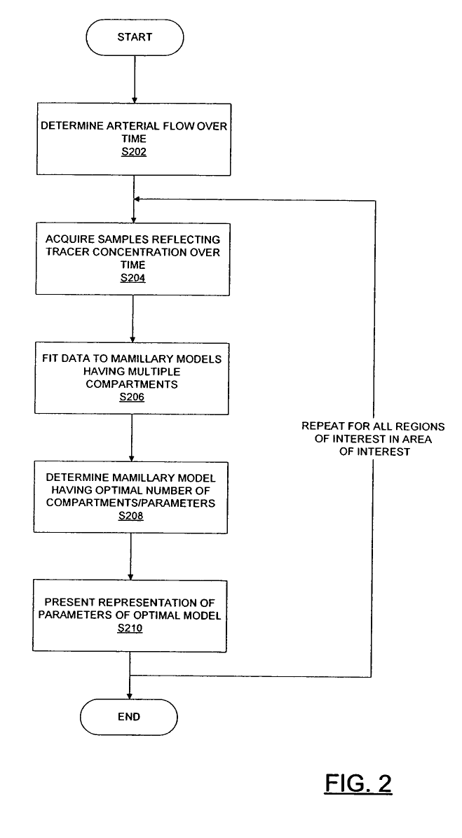

Specifically, steps performed by a DCE imaging system, after injection of a tracer into the blood stream are illustrated in FIG. 2. As illustrated, DCE imaging system acquires samples indicative of the amount of tracer in a region of interest, over time in step S204. Typically, the region of interest is the smallest sampling volume to be resolved by the DCE imaging system and is referred to as a volume-pixel (voxel). However, the region of interest may be larger than smallest sampling volume, and may vary in size or be adjustable among DCE imaging systems. Concurrently, the flow of blood at the arterial fluid source may be measured or approximated in step S202.

Once samples are acquired, the acquired data is fitted to the model(s) defined by equation (3) in step S206. Conventional data fitting techniques may be used. Models with increasing numbers of compartments j=1,2,3 . . . are fitted until the further increase does not materially affect the goodness of fit. That is

$${χ}^{2} = {\sum }_{k = 1}^{n} {[ \frac{{C}_{tiss}^{exp t} - {C}_{tiss} ( F , {t}_{1} {k}_{ij} , … )}{{var}_{k}} ]}^{2}$$

(equation (11)) for each j compartment model may be calculated, and the comparison of the goodness of fit between models having different numbers of compartments may be made using equation (12).

The form of each model having j=2, 3, 4 . . . compartments may be pre-calculated and stored at the DCE device, as a result of the pre-loaded software.

Ultimately, a preferred model having m compartments is used. In the disclosed embodiment, the number of compartments, m, of this model is chosen so that the goodness of fit between the m^{th} and (m+1)^{st} compartment model is statistically insignificant.

For example, using equation (12), the resulting value F* may be compared with the F* associated with a significance of α=0.05. We note that the Chi-square for a more complex model should be less than the Chi-square for reduced model.

Once the preferred model is assessed, values of the parameters for that model for the fitted region of interest may be graphically presented in step S208. Typically, parameter values including (F,t₁, kᵢⱼ . . . ) have already been calculated in the fitting step S206.

Indicators of the values of the parameters may be presented in any number of ways. For example, F for region of interest may be presented. Values derived from the measured parameters may be similarly presented. For example,

$${v}_{i} = {v}_{1} \frac{{k}_{i 1}}{{k}_{1 i}}$$

(equation (16)) or E (equation (15)) for each region of interest could be presented instead of, or in addition to other parameters.

For example, each region of interest may be graphically presented as a pixel or group of pixels on a colour display in a colour that varies in dependence on the value of an associated parameter. The brightness of the pixel in that colour may be varied in dependence on the value of the parameter. For multiple parameters, an image representative of each parameter may be presented.

An operator may choose between possible images (each representing the value of one parameter) through user input, such as by pressing of a key, or the like. Alternatively, each region of interest could be represented by multiple pixels, with the colour of one representative pixel varying in dependence of one of the multiple parameters. Of course, values of parameters for the model could be presented in any number of ways understood by a person of ordinary skill. The parameter values could be presented numerically, etc.

Steps S204 to S210 may be repeated for all regions of interest within a broader area. For example, steps S204 to S210 could be repeated for an area corresponding to the total resolution of the display. Alternatively, steps S204 to S210 could be repeated for subsequent regions of interest, using a model with the same number of compartments as for the first region of interest and for each other region of interest within the area.

In an alternate embodiment, models defined by equation (18) may be used in place of or in addition to models defined by equation (3) in steps S204 to S210. So, an optimal model from those defined by equation (18) may be chosen in step S208. Alternatively, an optimal model from models defined by equation (3) and (18) may be selected. The preferred model will have the least number of parameters without having a goodness of fit significantly worse than that of models with higher numbers of parameters. A more complex model can thus mean either an additional compartment in the mamillary model, or the inclusion of an additional parameter required by the capillary distribution. To test whether the difference is significant, a one-tailed F-test at significance level α=0.05 can be implemented, such that the decision rule is:
If f≦F*(1-α; γ_{DP}-γ_{DCDP}; γ_{DCDP}), distributed parameter model is adequate.
If f>F*(1-α; γ_{DP}-γ_{DCDP}; γ_{DCDP}), distributed parameter model with capillary distribution is justifiable.

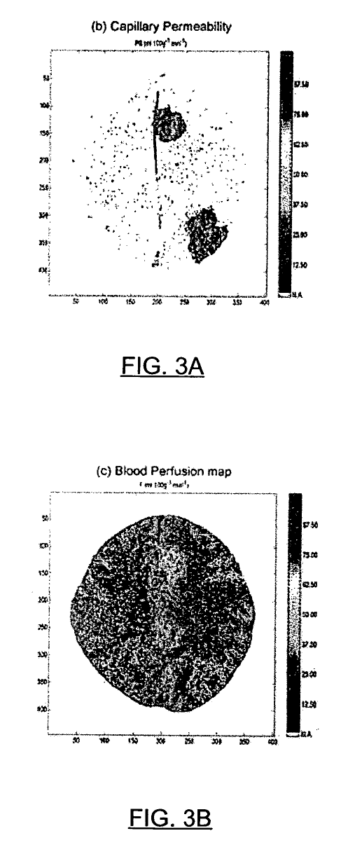

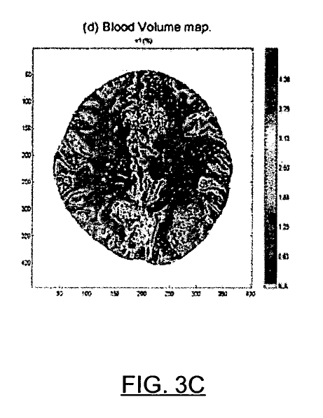

Conveniently, capillary permeability, blood perfusion and blood volume maps may thus be selected for an imaged area upon user selection. Example capillary permeability, blood perfusion and blood volume maps for the same area of interest are depicted in FIGS. 3A-3C, respectively.

These maps may be useful in detecting disease or physiological or functional damage to cell areas. For example, as will be appreciated by a person of ordinary skill, tumor regions may be detecting in regions of high permeability (PS).
An example: Application to a prostate case study

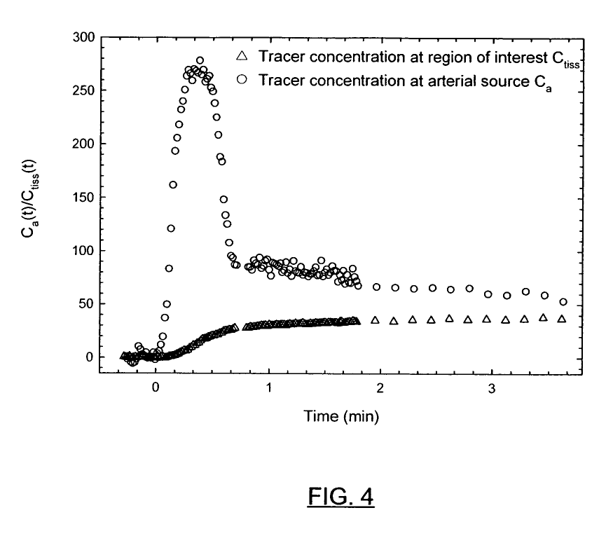

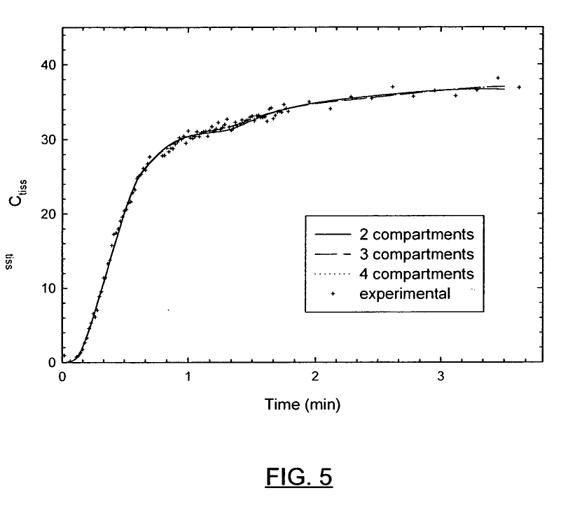

It is instructive to apply the present mamillary model to a tumor study case to further illustrate some physiological aspects of the model, and also for comparison with another model. The arterial input function and tissue concentration-time curve of this study case are shown in FIG. 4. Cₐ(t) was sampled at the iliac arteries simultaneously in the same CT scan as the prostate tissue concentration-time curve, Cₜᵢₛₛ(t). The contrast medium used was the x-ray dye lopamidol and the region of interest for Cₜᵢₛₛ was drawn over the prostate gland. Example imaging and patient protocols that were used are, for example, expounded upon in T. S. Koh, V. Zeman, J. Darko, T. Lee, M. F. Milosevic, M. Haider, P. Warde, and I. W. T. Yeung, “The inclusion of capillary distribution in the adiabatic tissue homogeneity model of blood flow”, Phys. Med. Biol., vol. 46, pp. 1519-1538, 2001.

The ‘goodness-of-fit’ between model and data can be given by the χ² statistic as detailed in equation (11).

A 2-compartment distributed-parameter model has 4 adjustable parameters (i.e F, t₁, k₁₂ and k₂₁), and each additional compartment introduces two additional parameters given by the rate constants.

The tissue concentration-time curve Cₜᵢₛₛ(t) was sampled at a region of interest over the prostate tumor. To test whether the observed tumor tissue data admits additional compartments, thereby demonstrating tissue kinetic heterogeneity, the tumor data set is fitted to models with an increasing number of compartments. An adequate model would be one that is the simplest and whose goodness-of-fit is not significantly worse than the more complex ones. A test statistic to compare the goodness-of-fit of a more complex model (χ²_{c}) with that of a less complex (reduced) model (χ²ᵣ) can be given by equation (12), namely

$${F}^{*} ( {χ}_{r}^{2} - {χ}_{c}^{2} ) = \frac{{χ}_{r}^{2} - {χ}_{c}^{2}}{d {f}_{r} - d {f}_{c}} / \frac{{χ}_{c}^{2}}{d {f}_{c}}$$

which follows the F-distribution. The degrees of freedom dfᵣ and df_{c} are those associated with the reduced and complex models, respectively. If χ²_{c} is not much less than χ²ᵣ, then the complex model does not account for much more of the variability of the experimental data than does the reduced model, in which case the data suggest that the reduced model is adequate. Correspondingly, large values of F* would justify the more complex model.

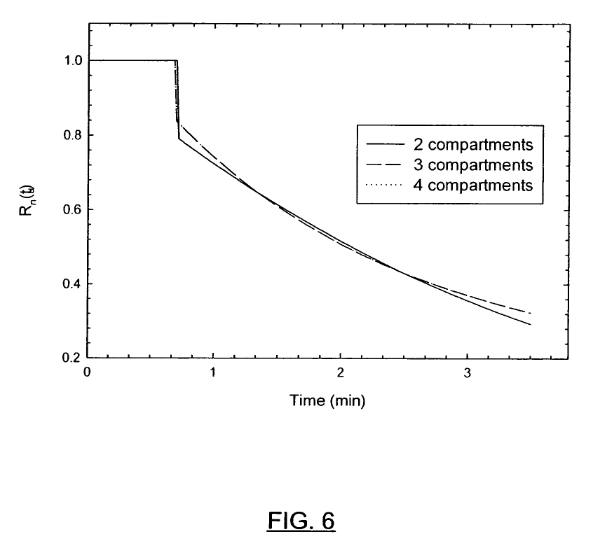

The fitted Cₜᵢₛₛ(t) curves using 2-, 3- and 4-compartment distributed-parameter models are shown in FIG. 5 and their corresponding impulse residue functions are shown in FIG. 6. The values for the parameters adjusted in the model fittings together with their error estimates (as percent CVᵢ) are listed in Table 1.

TABLE 1

Number of Compartments

| Parameter | 2 | 3 | 4 |
|---|---|---|---|
| F (ml min⁻¹ g⁻¹) | 0.213 (1) | 0.212 (5) | 0.212 (6) |
| t₁ (min) | 0.717 (3) | 0.688 (9) | 0.688 (11) |
| K₂₁ (min⁻¹) | 2.183 (4) | 1.854 (15) | 1.853 (17) |
| K₁₂ (min⁻¹) | 0.744 (4) | 1.581 (17) | 1.579 (20) |
| K₃₁ (min⁻¹) | | 0.818 (25) | 0.816 (32) |
| K₁₃ (min⁻¹) | | 0.259 (28) | 0.264 (36) |
| K₄₁ (min⁻¹) | | | 0.000 (44) |
| K₁₄ (min⁻¹) | | | 0.000 (53) |

It is worthwhile to note that the estimated F and t₁ values remain relatively unchanged even with the inclusion of additional compartments, and that these estimated values are consistent with those obtained using the adiabatic tissue homogeneity model as detailed in T. S. Koh et al., supra.

The similarities in the parameter estimates obtained using the present 3- and 4-compartment models (differing only at the second and third decimal places) and near zero values of k₁₄ and k₄₁ (non-zero only at the fourth decimal place) are indicative of the fact that the present data set could not resolve an additional fourth compartment. The goodness-of-fit estimates for the model fittings are respectively, χ²=149.5, 130.9 and 130.9 for the 2-, 3-, and 4-compartment models. The χ² values for the 3- and 4-compartment models differ only at the second decimal place and the corresponding F* value is near zero, which rules in favour of the 3-compartment model. The task now is to determine whether the 3-compartment model could be justified based on its χ² value. Comparing the χ² values for the 2- and 3-compartment models, and for a significance level of 0.05, we require F(0.95; 2; 105)≈3.1. Since F*(149.5, 130.9)=7.4>3.1, we may conclude that the 3-compartment model is justifiable over the 2-compartment model, and that it is adequate for the present data set.

These results again suggest two kinetically distinct peripheral tissue compartments in exchange with the vascular compartment, which is consistent with recent findings of kinetic heterogeneity in tumors. It has been hypothesized that the fast extravasation component can be identified to describe the filling of viable tissue; and the slow extravasation constant describes exchange with an additional interstitial compartment, such as necrotic tissue.

The example serves to illustrate the potential applicability of the present mamillary distributed-parameter model for studying kinetic heterogeneity in tumorous tissue.

Of course, the above described embodiments are intended to be illustrative only and in no way limiting. The described embodiments of carrying out the invention are susceptible to many modifications of form, arrangement of parts, details and order of operation. The invention, rather, is intended to encompass all such modification within its scope, as defined by the claims.

1. A method of dynamic contrast enhanced imaging, comprising:
for a region of interest, sampling the concentration of a tracer liquid in a fluid over a time interval to arrive at a plurality of samples;
fitting said plurality of samples to a plurality of multi-compartment mamillary pharmacokinetic models, each of said plurality of models modelling fluid flow in a number of interstitial compartments about a central compartment at said region interest, as a result of flow caused by a source of said fluid, so as to determine a plurality of fitting parameters for each of said plurality of models;
determining a preferred one of said plurality of models;
presenting indicators indicative of values of said plurality of parameters for said preferred one of said models.

2. The method of claim 1, wherein said determining further comprises calculating a goodness of fit to said plurality of samples for each of said plurality of models, and wherein said preferred one of said pharmacokinetic models is determined as said one of said plurality of plurality of pharmacokinetic models for which a goodness of fit is not significantly lower than a goodness of fit of others of said pharmacokinetic models having a number of compartments in excess of the number of compartments of said preferred pharmacokinetic model.

3. The method of claim 1, wherein said determining further comprises calculating a goodness of fit to said plurality of samples for each of said plurality of models, and wherein said preferred one of said pharmacokinetic models is determined as said one of said plurality of plurality of pharmacokinetic models for which a goodness of fit is not significantly lower than a goodness of fit of others of said pharmacokinetic models having a number of parameters in excess of the number of parameters of said preferred pharmacokinetic model.

4. The method of claim 1, wherein each of said pharmacokinetic models has an impulse residue response function that may be expressed in the form,
$$\begin{matrix}{R}_{n} ( t ) = {L}_{t}^{- 1} [ \frac{1}{s} ] - exp ( - {χ}_{n} ) {L}_{t}^{- 1} [ exp ( - {t}_{1} s ) \frac{1}{s} {\prod }_{i = 2}^{n} exp ( \frac{{k}_{i}}{s + {k}_{1 i}} ) ] \\ = u ( t ) - exp ( - {χ}_{n} ) u ( t - {t}_{1} ) {\int }_{0}^{t - {t}_{1}} ( {y}_{2} ⊗ … ⊗ {y}_{i} ⊗ … ⊗ {y}_{n} ) ⅆ τ . \\ wherein \\ {y}_{i} ( t ) = {L}_{t}^{- 1} [ exp ( \frac{{k}_{i}}{s + {k}_{1 i}} ) ] \\ = exp ( - {k}_{1 i} t ) {δ}_{t} ( t ) + exp ( - {k}_{1 i} t ) \sqrt{\frac{{k}_{i}}{t}} {I}_{1} ( 2 \sqrt{{k}_{i} t} ) ;\end{matrix}$$
I₁ is the modified Bessel function; u(t) is the Heaviside unit step function, δₜ is the dirac delta function; n is the number of compartments for said model; k₁ⱼ is a transfer constant denoting the rate of permeation from the central compartment to the nth interstitial compartment, per unit volume of the capillary,
$${χ}_{n} = {t}_{1} {\sum }_{i = 2}^{n} {k}_{i1} , and {k}_{i} = {t}_{1} {k}_{1 i} {k}_{i1} .$$

5. The method of claim 1, wherein said indicators include indicators of capillary permeability in said region.

6. The method of claim 1, wherein said indicators include indicators of blood perfusion in said region.

7. The method of claim 1, wherein said indicators include indicators of blood volume in said region.

8. The method of claim 1, wherein said indicators include indicators of the fractional extraction constant from said central compartment to said interstitial compartments in said region.

9. The method of claim 1, wherein said presenting comprises presenting coloured pixels having colours reflecting values of said indicator.

10. The method of claim 9, further comprising repeating the steps of sampling, fitting, determining and displaying for multiple regions of interest in an area of interest.

11. The method of claim 1, further comprising,
for further regions of interest, sampling the concentration of said tracer liquid in said fluid over a time interval to arrive at a plurality of samples;
fitting said plurality of samples for each further region of interest to said preferred one of said multi-compartment mamillary pharmacokinetic models, so as to determine a plurality of fitting parameters for said plurality of models for each of said further regions of interest;
presenting indicators indicative of values of said plurality of parameters for said preferred one of said multi-compartment mamillary pharmacokinetic models for said further regions.

12. The method of claim 1, wherein said sampling is performed by magnetic resonance imaging.

13. A method of dynamic contrast enhanced imaging, comprising:
for a region of interest, sampling the concentration of a tracer liquid over a time interval to arrive at a plurality of samples;
fitting said plurality of samples to a multi-compartment mamillary pharmacokinetic model modelling fluid flow in a number of interstitial compartments about a central compartment at said region interest, as a result of flow caused by a source of said fluid , said pharmacokinetic model having an impulse residue response function that may be expressed in the form,
$$\begin{matrix}{R}_{n} ( t ) = {L}_{t}^{- 1} [ \frac{1}{s} ] - exp ( - {χ}_{n} ) {L}_{t}^{- 1} [ exp ( - {t}_{1} s ) \frac{1}{s} {\prod }_{i = 2}^{n} exp ( \frac{{k}_{i}}{s + {k}_{1 i}} ) ] \\ = u ( t ) - exp ( - {χ}_{n} ) u ( t - {t}_{1} ) {\int }_{0}^{t - {t}_{1}} ( {y}_{2} ⊗ … ⊗ {y}_{i} ⊗ … ⊗ {y}_{n} ) ⅆ τ \\ wherein \\ {y}_{i} ( t ) = {L}_{t}^{- 1} [ exp ( \frac{{k}_{i}}{s + {k}_{1 i}} ) ] \\ = exp ( - {k}_{1 i} t ) {δ}_{t} ( t ) + exp ( - {k}_{1 i} t ) \sqrt{\frac{{k}_{i}}{t}} {I}_{1} ( 2 \sqrt{{k}_{i} t} ) ;\end{matrix}$$
I₁ is the modified Bessel function; u(t) is the Heavisid unit step function, δₜ is the dirac delta function; n is the number of compartments for said model, k₁ⱼ is a transfer constant denoting the rate of permeation from the central compartment to the nth interstitial compartment, per unit volume of the capillary,
$${χ}_{n} = {t}_{1} {\sum }_{i = 2}^{n} {k}_{i1} , and {k}_{i} = {t}_{1} {k}_{1 i} {k}_{i1} ;$$
displaying indicators indicative of values of said multiple parameters for said model.

14. A dynamic contrast enhanced imaging device comprising software adapting said device to perform the method of claim 1.

15. Computer readable medium storing software that when loaded at a dynamic contrast enhanced imaging device, adapts said device to perform the method of claims 1.

16. A method of dynamic contrast enhanced imaging, comprising:
for a region of interest, sampling the concentration of a tracer liquid over a time interval to arrive at a plurality of samples;
fitting said plurality of samples to a multi-compartment mamillary pharmacokinetic model modelling fluid flow in a number of interstitial compartments about a central compartment at said region interest, as a result of flow caused by a source of said fluid, said pharmacokinetic model, modelled as a function of time that may be expressed in the form,
$$\begin{matrix}{H}_{n} ( t ) = {H}_{n}^{v} ( t ) + {H}_{n}^{p} ( t ) , \\ wherein \\ {H}_{n}^{v} ( t ) = 1 - {\int }_{0}^{t} ⅆ {t}_{1} g ( {t}_{1} ) \\ and \\ {H}_{n}^{p} ( t ) = {\int }_{0}^{t} ⅆ {t}_{1} g ( {t}_{1} ) {R}_{n}^{p} ( t , {t}_{1} ) \\ and wherein \\ {\int }_{0}^{∞} g ( τ ) ⅆ τ = 1 ; \\ {R}_{n} ( t ) = {L}_{t}^{- 1} [ \frac{1}{s} ] - exp ( - {χ}_{n} ) {L}_{t}^{- 1} [ exp ( - {t}_{1} s ) \frac{1}{s} {\prod }_{i = 2}^{n} exp ( \frac{{k}_{i}}{s + {k}_{1 i}} ) ] \\ = u ( t ) - exp ( - {χ}_{n} ) u ( t - {t}_{1} ) {\int }_{0}^{t - {t}_{1}} ( {y}_{2} ⊗ … ⊗ {y}_{i} ⊗ … ⊗ {y}_{n} ) ⅆ τ \\ wherein \\ {y}_{i} ( t ) = {L}_{t}^{- 1} [ exp ( \frac{{k}_{i}}{s + {k}_{1 i}} ) ] \\ = exp ( - {k}_{1 i} t ) {δ}_{t} ( t ) + exp ( - {k}_{1 i} t ) \sqrt{\frac{{k}_{i}}{t}} {I}_{1} ( 2 \sqrt{{k}_{i} t} ) ;\end{matrix}$$
I₁ is the modified Bessel function; u(t) is the Heaviside unit step function, δₜ is the dirac delta function; n is the number of compartments for said model, k₁ⱼ is a transfer constant denoting the rate of permeation from the central compartment to the nth interstitial compartment, per unit volume of the capillary,
$${χ}_{n} = {t}_{1} {\sum }_{i = 2}^{n} {k}_{i1} , and {k}_{i} = {t}_{1} {k}_{1 i} {k}_{i1} ;$$
displaying indicators indicative of values of said multiple parameters for said model.
